(19) United States
(12) Patent Application Publication
(10) Pub. No.: US 2011/0262935 A1
(43) Pub. Date: Oct. 27, 2011

(54) METHOD FOR DETECTING A PRION INFECTION

(75) Inventors: Aude Arzel, Montrouge (FR); Bruno You, L'Hay Les Roses (FR)

(73) Assignee: LGB-Biotecnologies, Les Ullis (FR)

(21) Appl. No.: 13/141,483

(22) PCT Filed: Dec. 22, 2009
(86) PCT No.: PCT/FR2009/052656

§ 371 Date: Jun. 22, 2011

(30) Foreign Application Priority Data
Dec. 22, 2008 (FR) 0858968

(51) Int. Cl. G01N 33/53; C12Q 1/37

(52) U.S. Cl. 435/7.1; 435/23

(57) ABSTRACT

The invention relates to an in vitro method for detecting and/or titrating an unconventional transmissible agent (UTA) or a protein of pathological conformation which is a marker for the infectivity of the UTA, in a sample, comprising a step of concentrating the sample before and after digestion with a protease.

The present invention relates to an optimized method for detecting the PrPsc prion protein by Western blotting, and also to the use thereof, in particular for evaluating and/or testing, in vitro, the effectiveness of a process for obtaining or treating a biological product, or for evaluating and/or testing, in vitro, a decontamination procedure and/or for evaluating or screening compounds that may possess a modulatory activity on prion infectivity.

### PRIOR ART

Transmissible spongiform encephalopathies (TSE) group together a set of genetic or acquired diseases characterized by degeneration of the central nervous system (CNS). The most common form in humans is Creutzfeldt-Jakob disease (CJD), but TSEs also exist in many mammals (in particular sheep scrapie and bovine spongiform encephalopathy). The etiological agent of these diseases is classified in the category of “unconventional transmissible agents” (UTA). The signature of the disease is the presence of an extracellular protein, called prion protein (PrP), which is converted, during the disease, into an insoluble form that is resistant to proteases such as proteinase K, and which accumulates in the central nervous system. This pathological abnormal form of PrP, called PrPsc, is copurified with the infectivity and its accumulation precedes the appearance of histological lesions. It is the result of a modification in the conformation of the PrP prion protein. There has been no demonstration of any modification of the expression of the gene encoding PrP, nor any detrimental modification of its translation (Prusiner, Biochemistry 1992; 31:12277-88).

The data currently available do no make it possible to demonstrate that the transmissible agent responsible for TSEs is present in an infecting form in blood derivatives (Brown et al., 2001, Semin Hematol.; 38(4 Suppl 9):2-6).

However, it cannot be concluded that it is absent, this uncertainty resulting, firstly, from the probable very low concentration in the blood and, secondly, from the very long clinically silent period of incubation characteristic of these diseases, which precedes the appearance of the clinical signs.

In addition, the exceptional resistance of UTAs prevents any recourse to inactivity methods conventionally used, such as Tween-TNBP solvent/detergent treatment, which are proven to be effective for reducing the viral load of blood derivatives, such as cryoprecipitated plasma proteins (factor VIII, from Willebrand factor, etc.). Given that the obtaining or the treatment of biological products, such as plasma coagulation proteins, must incorporate viral elimination/inactivation steps for the purpose of therapeutic use, the pharmaceutical blood-derived medicament industry is today seeking to evaluate the theoretical risk of transmission of variant CJD by blood-derived products.

Currently, the method for titrating UTA-related infectivity conventionally implemented uses a method of in vivo titration in animals, such as the golden hamster, by intracerebral injection of various dilutions of a UTA-loaded test product. According to the number of animals affected in the various groups corresponding to the dilutions carried out, it is possible to calculate an infectious titre and to establish the reduction factor of a given method based on an untreated reference. However, this method has the drawback of being long (approximately one year), expensive and not very compatible with an industrial-scale development for which it is desired to rapidly ascertain the effectiveness with respect to elimination of the prion.

In addition, it is often necessary to introduce a step of concentrating the infectious agent so as to increase the sensitivity of the titration methods. All the procedures for concentrating infectious agents responsible for TSE today involve purification of PrPsc.

Other methods have been proposed for the in vitro titration of TSE infectious agents.

The techniques for detecting PrPsc by Western blotting (Mac Gregor, Transfusion J. Medecine 2001; 11, 3-14) or by ELISA generally require prior digestion of the sample to be analysed with proteinase K, or denaturation with chaotropic agents in order to distinguish the pathological protein (PrPsc) from the normal protein (PrP).

Another titration method has recently been developed based on the use of PrPsc-specific antibodies which do not recognize PrP (Korth et al, Nature 1997 Nov. 6, 390 (6655): 74-7).

U.S. Pat. No. 6,150,583 describes, for its part, the production of transgenic animals expressing a PrP labelled with a heterologous epitope which is possibly exposed at the surface of the prion protein, depending on the conformation of the latter.

The method called “PMCA” (protein misfolding cyclic amplification), described in the document Saborio et al. (Nature; 2001, 411, 810-3), envisages bringing pathological forms originating from a tissue or a fluid derived from a contaminated animal into contact with a nonpathological form of the UTA protein, in order to convert said protein into a pathological form. The PMCA method, although still under development, is proven to be at least as sensitive as the bioassay. Nevertheless, it provides no evidence as to the infectivity of the PrPsc detected and is found to be difficult to carry out with plasma matrices. In addition, an uncertain reproducibility and false-positive results have been reported.

Document WO 2005/022148 describes a method for in vitro titration, called “TCIA” (tissue culture infectivity assay), of a UTA in a biological product, by means of bringing transgenic cells which withstand the replication of said UTA into contact with the biological product, and then culturing these cells for one or more passages in order to amplify the amount of UTA present in the biological product by replication of UTA.

### SUMMARY OF THE INVENTION

The invention now provides an optimization of the detection of PrPsc by Western blotting and, consequently, of the quantification of PrPsc in a sample by Western blotting.

The inventors have in fact demonstrated that it is possible to concentrate the PrPsc in a sample, before and after the proteinase K digestion step.

More generally, the invention relates to an in vitro method for detecting and/or titrating an unconventional transmissible agent (UTA) or a protein of pathological conformation which is a marker for the infectivity of the UTA, in a sample, comprising a step of concentrating the sample before and after digestion with a protease.

Thus, a subject of the invention is an in vitro method for detecting and/or titrating an unconventional transmissible agent (UTA) or a protein of pathological conformation which is a marker for the infectivity of the UTA, in a sample, comprising the steps consisting in:

i) concentrating said UTA or said infectivity-marker protein present in the sample,
ii) subjecting the concentrated sample to digestion with protease, preferably proteinase K,
iii) concentrating the digest obtained in step (ii),
iv) detecting and/or titrating said UTA or said infectivity-marker protein in the concentrated digest obtained in step (iii).

Preferably, the protein of pathological conformation, which is a marker for UTA, is the PrPsc prion protein.

Advantageously, the concentrating step (i) is carried out by centrifugation, preferably by ultracentrifugation.

In one preferred embodiment, the concentrating step (i) comprises an ultracentrifugation of from 100 000 g to 140 000 g, preferably at 140 000 g, for 1 hour.

Advantageously, the concentrating step (iii) is carried out by centrifugation.

Preferably, step (iv) is carried out by immunochemistry, more particularly by Western blotting.

The method may preferably comprise an intermediate step of diluting after step (iii) of concentrating the digest, and before detecting or titrating step (iv).

The sample is preferably chosen from the group constituted of blood products and derivatives thereof, foods, and cosmetic products.

According to one particular embodiment, the concentrating of the PrPsc before the digesting step is obtained by ultracentrifugation of the sample to be titrated at preferably 140 000×g for 1 hour at ambient temperature, with a sample volume of approximately 8.3 ml introduced into a tube pretreated for 1 hour with bovine serum albumin at 0.1%, and with a swinging rotor. At the end of this ultracentrifugation, the sedimented pellet is taken up preferably in a volume of water or other buffer of preferably between 20 and 60 μl.

The pellet suspension is incubated in the presence of proteinase K and under the conditions optimal for the digestion.

At the end of the digesting step, the concentrating of the PrPsc is obtained by further centrifugation of the digestion product, preferably at 18 000×g for 30 minutes.

The invention is also directed towards an in vitro method for detecting and/or titrating the immunoreactivity of an unconventional transmissible agent (UTA) of which the marker is a protein of pathological conformation, in a sample.

Another subject of the invention is an in vitro method for evaluating and/or testing a process for obtaining or treating a biological product or a material that may be contaminated with a UTA, in which method a titration method as defined above is applied to said biological product or material, (A) upstream and (B) downstream of said process, and the two titre values (A) and (B) obtained are compared.

The invention is also directed towards an in vitro method for evaluating and/or testing a procedure for decontamination of a biological product or of a material, in which method a titration method as defined above is applied to said biological product or material, (A) upstream and (B) downstream of said procedure, and the two titre values (A) and (B) obtained are compared.

Yet another subject of the invention is an in vitro method for evaluating a compound capable of affecting the immunoreactivity of an infectious biological product, in which method a titration method as defined above is applied to said infectious biological product, (A) in the presence and (B) in the absence of said compound to be evaluated, and the two titre values (A) and (B) obtained are compared.

Yet another subject of the invention is an in vitro method for diagnosing a transmissible spongiform encephalopathy in a human individual or a non-human animal individual, which comprises detecting the presence, in a biological sample from said individual, of an unconventional transmissible agent (UTA) which is a protein of pathological conformation, by means of the method as defined above.

### DETAILED DESCRIPTION OF THE INVENTION

### Definitions

A “PrP prion protein” is generally a sialoglycoprotein anchored to the plasma membrane via a phosphatidyl glycolipid (GPI), naturally present in the cells and involved in their normal functioning. The normal form of the protein, i.e. the nonpathological form, is generally called PrPc. It is involved in the development of the nervous system in the embryo. In adults, it is expressed essentially in the brain and the spinal cord (neurons and glia). It is involved in cell differentiation and adhesion processes. It also appears to have an antioxidant protective role with respect to programmed cell death (apoptosis). This protein also appears to have a role in the folding of other proteins.

The “pathological” form, PrPsc, of PrP generally denotes an isoform of the nonpathological protein. It represents the marker for prion diseases. This modified three-dimensional structure confers atypical physicochemical properties on the prion protein, which are reflected by a greater resistance to the usual means of disinfection and sterilization (heat, chemical products, enzymes, etc.). The pathological prion protein thus acquires self-aggregation capacities and can thus form deposits, in particular in the brain, causing neuron death. The agent responsible for replication or propagation of the pathological prion protein appears to be the pathological prion protein itself, since it is capable of “propagating or multiplying exponentially”, deforming the healthy prion proteins into pathological prion proteins. The “pathological” form of PrP is therefore the form of the protein of which the conformation is correlated with the appearance of a TSE in the infected human or non-human animal.

The “titre”, expressed in Western blotting units, is an arbitrary value determined by the limiting dilution of the sample starting from which no immunoreactivity is any longer observed by Western blotting.

In the context of the invention, the term “UTA” represents any unconventional transmissible agent, such as those responsible, in humans, for familial or sporadic CJD, for Kuru disease or for variant CJD, or alternatively those responsible, in animals, for natural TSEs, such as ovine scrapie, bovine or feline spongiform encephalopathy, chronic wasting disease in cervids or spongiform encephalopathy in mink, or, finally, TSE strains experimentally adapted to laboratory animals. In the context of the invention, the UTA is also denoted by the term “infectious agent”.

The term “sample” denotes any source of material which may be contaminated with a UTA. Such a source of material may, for example, be a liquid, a food product, a drink, a cosmetic product or a product derived from genetic engineering, a molecule capable of modulating the infectivity of a UTA, this list not being limiting. Preferably, it is a biological sample, for example a biological fluid or a tissue or tissue extract. Such a tissue may be a brain tissue, a vertebral column tissue or a tonsil tissue, this list not being limiting. The sample may also be a composition derived from a human or animal source, such as growth hormones or cell extracts, for instance pituitary extracts. Such a composition may in fact be contaminated with a UTA. In the case of a biological fluid, the latter may be blood, lymph, urine or milk, this list not being limiting. Preferentially, the sample is a blood product or a derivative, for example a plasma derivative or a plasma protein concentrate.

Protein Concentration:

The PrPsc protein can be concentrated by means of a first step of ultracentrifugation, preferably at 140 000×g for 1 hour at ambient temperature. Advantageously, the concentrating is obtained using a tube pretreated with bovine serum albumin at 0.1% for 1 hour at ambient temperature or overnight at +4° C. This treatment consists in neutralizing the electrostatic forces of the material (polymer) of the tube capable of attaching to the walls of the tube the PrPsc present in the sample to be analysed. This treatment is obtained by filling the ultracentrifugation tube with the BSA solution and leaving it to incubate as described above.

At the end of the ultracentrifugation, the supernatant is removed and the pellet is taken up with a volume of preferably 50 μl of water or of any other buffer.

Elimination of the Nonpathological Prion Protein (PrPc Form):

The elimination of the nonpathological form (PrPc) in the sample can be carried out on the basis of biochemical properties of the PrPsc which make it possible to distinguish it from the nonpathological proteins, in particular the fact that the PrPsc is relatively more resistant to protease-based treatments and is less soluble, or even insoluble, even in the presence of detergents. Thus, a treatment with protease, for example proteinase K, is used, for example, to eliminate the sensitive forms (PrPc) from the forms less sensitive to digestion (PrPsc).

In one particular embodiment, a proteinase K digestion is carried out which results in the digestion mainly of the PrPc, with little or no digestion of the PrPsc. It is in fact a property of the PrPsc that is more resistant to PK compared with PrPc. The subsequent detecting step therefore no longer detects the nonpathological form of the protein since it has been digested by the protease.

Concentration of the Digested PrPsc Form:

The pathological prion protein “resistant” to the proteinase treatment can be concentrated by centrifugation, preferably at 18 000×g for 30 minutes at ambient temperature. At the end of the step of centrifuging the sample digested with proteinase K, the supernatant is removed and the pellet is taken up in a volume of loading buffer corresponding to the maximum volume of a comb well for loading on an electrophoresis gel (typically 10 to 15 μl) or 25 μl if a dilution range in loading buffer, and preferably a 3-fold stepwise dilution, is envisaged.

The nature of the loading buffer is determined by the PrPsc detection method subsequently used (see next paragraph). Thus, for PrPsc detection by Western blotting, the loading buffer is preferentially Laemmli buffer (typically: 0.15M Tris-HCl, pH=6.8, 2 to 10% SDS, 0.06M DTT, 0.3% bromophenol blue, 11.5% glycerol).

Detection of the UTA Proteins:

The step of detecting PrPsc in the sample digested and concentrated as described above can be carried out by means of the following methods: immunochemical methods such as Western blotting or ELISA, immunoblotting, or radioactivity assay, electron microscopy, turbidimetry assay for detecting aggregates, and also structural tests including NMR (nuclear magnetic resonance), circular dichroism, Raman spectroscopy, UV absorption, this list not being limiting.

UTA Titration:

The detection of the pathological form of the PrP can be combined with a determination of the amount of PrPsc present in the sample.

In one embodiment of the invention, the suspension of digested and concentrated sample is diluted in loading buffer according to a geometric progression also termed stepwise dilution. The latter is preferably 3-fold. Each dilution point is then subjected to a test for detecting the UTA proteins as described above.

Arbitrarily, the PrPsc Western blotting unit is defined as the inverse of the first negative dilution not exhibiting any signal specific for the PrPsc (PrPsc extinction signal) in the volume of sample not diluted and used for the detection test.

The method of the invention makes it possible to quantify the UTA-related immunoreactivity over a theoretically infinite range. In one embodiment of the invention, the method made it possible to quantify the immunoreactivity related to the scrapie strain over a range of approximately 7.5 log₁₀, i.e. approximately 31 000 000 Western blotting units. This can, for example, make it possible to meet the criteria for validation of the effectiveness of the processes for obtaining biological products with respect to the elimination of UTAs.

Applications of the Method for Detecting or Titrating the UTA-Related Immunoreactivity:

The invention also relates to the application of the titration method according to the invention to a method for evaluating and/or testing, in vitro, a process for obtaining or treating a biological product that may be contaminated with a UTA. This evaluating and/or testing method is characterized in that a method of titration according to the invention, as described above, is applied to the biological product, upstream and downstream of said process, and in that the two titre values obtained are compared. The degree of elimination of the UTA or the UTA reduction factor is determined by comparison between the two measurements.

In particular, the titration method according to the invention has the ability to be readily applied to any type of process for obtaining or purifying biological products, in particular blood products, such as blood plasma derivatives, using for example chromatographies or nanofiltration.

Thus, the implementation of the method of the invention makes it possible to evaluate and/or test the effectiveness of a process (or of a part of a process) for obtaining or treating, or even purifying, any biological product that may be contaminated with a UTA, in the elimination of this UTA, by virtue of a titration using the titration method according to the invention. The amounts of UTA upstream and downstream of the process (or of the part of the process) for which it is desired to assess the effectiveness with regard to UTA are measured. The degree of elimination of the pathogenic agent is determined by comparison of the two measurements. Thus, the implementation of the present method can be carried out during a process for obtaining a biological product or in the context of a treatment for eliminating the UTA following the obtaining of the biological product.

The invention also relates to the application of the titration method according to the invention to a method for evaluating and/or testing, in vitro, a procedure for decontamination of a material. In this case, the UTA titre of a biological product containing a UTA is determined by means of the titration method according to the invention. This infected biological product is subsequently brought into contact with the material to be decontaminated and then the decontamination procedure is applied to this material. Finally, the titre of the biological product having undergone the decontamination procedure is again determined. The two titre measurements carried out upstream and downstream of the decontamination procedure are compared in order to evaluate the effectiveness of the decontamination procedure. The material may, for example, be a purification material, in particular a chromatography column, or alternatively be the sanitization of a chromatography column using sodium hydroxide.

The invention also relates to the application of the titration method according to the invention to a procedure for selecting and/or a method for evaluating a compound making it possible to reduce the titre of the infectious material. In this case, the UTA titre of a biological product containing a UTA is determined by means of the titration method according to the invention. This infected biological product is subsequently brought into contact with the test compound and then the titre of the biological product having undergone the decontamination procedure is again determined. The two titre measurements carried out upstream and downstream of bringing the sample into contact with the test compound are compared.

The invention also relates to the application of the titration method according to the invention to a procedure for selecting and/or a method for evaluating a compound capable of modulating the infectivity of a UTA. In this case, the UTA titre of a biological product containing a UTA is determined, in the presence and then in the absence of the compound to be evaluated, by means of the titration method according to the invention. The modes for bringing into contact with the compound are determined according to whether the action of the compound prevents the initiation of an infectious cycle or blocks an already initiated infectious cycle. In any event, the titre of the biological product is determined with and without treatment with the test product. The two titre measurements carried out are compared in order to evaluate the modulatory activity of the compound on the infectivity of a UTA.

The invention also relates to the application of the titration method according to the invention to a method for identifying a compound which makes it possible to modulate the conversion of the nonpathological form into the pathological form of the UTA, for example the conversion of PrP to PrP^{sc}. In this case, the UTA titre of a biological product containing a UTA is determined by means of the titration method according to the invention. This infected biological product is subsequently brought into contact with the test compound and then the titration method of the invention is applied in order to again determine the titre of the biological product. The two titre measurements carried out upstream and downstream of the bringing into contact with the compound are compared in order to evaluate the effectiveness of the test compound.

The method of the invention will be understood more clearly by means of the further description which follows, which does not limit the scope of the invention.

### EXAMPLES

### Materials and Methods

Stock of Ground Infected Hamster Brain Material Batch LN-6246 consisted of a microsomal fraction at 10% in PBS prepared from a brain of a hamster infected with the 263K strain. 3 aliquots called LN-6246-1, LN-6246-2 and LN-6246-3 were formed from batch LN-6246.

Batches LN-6246-A, LN-6246-B and LN-6246-C originated from a dilution of the sample LN-6246. This dilution was carried out by diluting 10⁶-fold, in PBS, an aliquot of LN-6246. The characteristics of the stocks of ground materials of brains from hamsters infected with the 263K strain used during the study are summarized in table I below.

TABLE I
Characteristics of the stocks of homogenates of
brains infected with the 263K strain

| | |
|---|---|
| Code | LN-6246 |
| Origin | Hamster infected with 263K |
| Biological tissue | Ground brain homogenate at 20% in |
| | serum containing glucose |
| Specific preparation | Microsomal fraction |
| Aliquots | 4 × 500 μl (A to D) |
| Storage | −80° C. +/− 10° C. |

Assaying of the PrPsc of the LN-6246 Samples by “Standard” Western Blotting

(I) Proteinase K Digestion Step:

20 μl of each of the samples (LN-6246-1, LN-6246-2 and LN-6246-3) were brought into contact with a mix consisting of 2 μl of digestion buffer and 2 μl of proteinase K enzyme. The suspension obtained was incubated at 48° C. for 40 minutes.

At the end of this incubation the following were successively introduced for each sample: 2 μl of a blocking buffer in order to stop the action of the proteinase K and then 20 μl of loading buffer (Laemmli buffer). The total reaction volume, called “digest” was then: 46 μl.

(II) Dilution Range Step:

For each sample, a 3-fold stepwise dilution range in loading buffer was prepared by dilution in cascade starting with 10 μl of digest to which 20 μl of loading buffer had been added. The digest obtained was said to be diluted to 1/3.

This dilution was reproduced with the digest diluted to 1/3, to which 20 μl of loading buffer had been added. The digest obtained then corresponded to the dilution to 1/9. This diluting operation was reproduced in cascade until dilution of the digest to 1/177181.

(III) Western Blotting Step:

For each of the points of the range of dilution of each of the digests obtained, a step of denaturation at 96° C. for 5 minutes was carried out. The denatured samples were then loaded, in a proportion of 10 μl, onto a gel for polyacrylamide gel electrophoresis under denaturing conditions (SDS-PAGE).

In the 10 μl of nondiluted digest loaded onto the lane of the gel, there was the equivalent of: (20 μl/46 μl)×10 μl=4.35 μl of the sample. This volume was called “initial Equivalent Volume” (VEq).

The proteins that had migrated in the gel were blotted onto a PVDF membrane by electroblotting. The PrPsc present on the membranes was detected by incubation with the 3F4 antibody and then a secondary antibody labelled with alkaline phosphatase (goat “anti-mouse antibody” antibody).

The labelled membranes were developed by chemiluminescence. A sample was considered to be positive if the electrophoretic profile with the three forms of glycosylated PrPsc was visible on the autoradiograms. Nevertheless, in the event of very low loads, only the predominant band corresponding to the diglycosylated form having a molecular weight of approximately 29 kDa was observed.

The titre of the samples LN-6246-1, LN-6246-2 and LN-6246-3 was calculated as follows:

T=(1/LD)×(1000/VEq)

### With:

LD: limiting dilution, i.e. the first dilution no longer exhibiting signals specific for the PrPsc on the autoradiogram.
VEq: initial Equivalent Volume, i.e. 4.354

Assaying of the PrPsc of the Samples LN-6246-A, LN-6246-B and LN-6246-C by “Optimized” Western Blotting According to the Invention:

3 ultracentrifugation tubes (Beckman ref: 361623) were filled with a solution of bovine serum albumin at 0.1% and incubated at +4° C. overnight (approximately 16 hours). At the end of the incubation, these tubes were emptied and filled with 8.3 ml of the sample LN-6246-A, LN-6246-B and LN-6246-C. These 3 tubes were ultracentrifuged with a swinging rotor (Beckman ref SW41) at 140 000×g for 1 hour at ambient temperature. At the end of the ultracentrifugation, the supernatant was removed and the pellets were taken up individually in 50 μl of water. The suspensions were called, respectively, LN-6246-A, LN-6246-B and LN-6246-C for the pellets taken up from the tubes LN-6246-A, -B and -C, respectively. The theoretical maximum concentration factor was therefore 8300/50, i.e. 166 times.

50 μl of each of the suspensions LN-6246-A, LN-6246-B and LN-6246-C were brought into contact with a mix consisting of 5 μl of digestion buffer and 5 μl of proteinase K enzyme. The suspension obtained was incubated at 48° C. for 40 min.

At the end of this incubation, 5 μl of a blocking buffer were introduced in order to stop the action of the proteinase K. The final volume of the “digest” was then: 65 μl.

For each of the digests obtained, concentrating was carried out by centrifugation at 18 000×g for 30 minutes at ambient temperature. At the end of centrifugation, the supernatant was removed and the pellet was taken up in 25 μl of loading buffer (Laemmli buffer).

A 3-fold stepwise dilution range in loading buffer was prepared by dilution in cascade starting with 10 μl of digest to which 20 μl of loading buffer had been added. The digest obtained was said to be diluted to 1/3.

This dilution was reproduced with the digest diluted to 1/3, to which 20 μl of loading buffer had been added. The digest obtained then corresponded to the dilution to 1/9. This diluting operation was reproduced in cascade until dilution of the digest to 1/729.

For each of the digest dilution range points obtained, a step of denaturation at 96° C. for 5 minutes was carried out. The denatured samples were then loaded, in a proportion of 15 μl, onto a gel for polyacrylamide gel electrophoresis under denaturing conditions (SDS-PAGE).

Under these operating conditions, the initial equivalent volume was: (8300/50)×(50 μl/65 μl)×(65 μl/25 μl)×15 μl=4980 μl for each of the samples LN-6246-A, LN-6246-B and LN-6246-C.

Compared with the titration method by “standard” Western blotting, the optimized method made it possible to increase the initial Equivalent volume by: 4980/4.32, i.e. a ratio of 1152 times, i.e. 3.06 log₁₀.

The proteins were separated by electrophoresis and analysed as described above in step III.

Results

The results of the titrations of the samples are summarized in table 1 below.

| | Method | | | | | |
|---|---|---|---|---|---|---|
| | | | | Optimized (according | | |
| | Standard | | | to the invention) | | |
| | | Titre | | Limit- | Titre | |
| | Limiting | (log10) | | ing | (log10) | |
| | dilution | WBu/ | Medium | dilution | WBu/ | Medium |
| Sample | (1/n) | ml | titre | (1/n) | ml | titre |
| LN-6246-1 | 177147 | 7.61 | 7.29 | NT | NA | |
| LN-6246-2 | 59049 | 7.13 | | NT | NA | |
| LN-6246-3 | 59049 | 7.13 | | NT | NA | |
| LN-6246-A | ND | <LoD | | 81 | 1.21 | 1.05 |
| LN-6246-B | ND | <LoD | | 27 | 0.73 | |
| LN-6246-C | ND | <LoD | | 81 | 1.21 | |

Legend:
NT: not tested,
ND: not detectable,
NA: not applicable,
LoD: limit of detection.

These data showed that the titre of the sample LN-6246-1, LN-6246-2 and LN-6246-3 could be determined with the standard method; this titre was, respectively: 7.61, 7.13 and 7.13 log₁₀ WBu/ml. The medium titre was 7.29 log₁₀ WBu/ml.

The titre of the samples LN-6246-A, LN-6246-B and LN-6246-C could not be determined with the standard method. Their titre was below the limit of detection of this method (theoretical detection limit of 2.8 log₁₀ WBu/ml).

On the other hand, with the optimized method, the titre of these samples could be determined. It was 1.21, 0.73 and 1.21 log₁₀ WBu/ml in the samples LN-6246-A, LN-6246-B and LN-6246-C, respectively. The medium titre of these samples was 1.05 log₁₀, WBu/ml.

The absence of detection of PrPsc in the samples LN-6246-A, LN-6246-B and LN-6246-C with the standard method was confirmed by the titre of these samples, which was below the limit of detection of the standard method. This level of titre could be determined only with the optimized method according to the invention.

### CONCLUSIONS

The method of assaying by optimized Western blotting, according to the invention, makes it possible to significantly lower the limit of detection of the Western blotting method. The steps of concentrating the PrPsc that are implemented in the optimized method in fact create an initial equivalent volume which is much higher than that measured with the standard method.

1. In vitro method for detecting and/or titrating an unconventional transmissible agent (UTA) or a protein of pathological conformation which is a marker for the infectivity of the UTA, in a sample, comprising a step of concentrating the sample before and after digestion with a protease.

2. Method according to claim 1, comprising the steps consisting in:
i. concentrating said UTA or said infectivity-marker protein present in the sample,
ii. subjecting the concentrated sample to digestion with a protease,
iii. concentrating the digest obtained in step (ii),
iv. detecting and/or titrating said UTA or said infectivity-marker protein in the concentrated digest obtained in step (iii).

3. Method according to claim 1, in which the protein of pathological conformation which is a marker for the UTA is the PrPsc prion protein.

4. Method according to claim 1, in which the step of concentrating before digestion is carried out by centrifugation, preferably by ultracentrifugation.

5. Method according to claim 4, in which the concentrating step (i) comprises ultracentrifugation at 140 000 g for 1 hour.

6. Method according to claim 2, in which the concentrating step (iii) is carried out by centrifugation.

7. Method according to claim 1, in which the protease is proteinase K.

8. Method according to claim 2, in which step (iv) is carried out by immunochemistry.

9. Method according to claim 8, in which step (iv) is Western blotting.

10. Method according to claim 2, comprising an intermediate step of dilution after step (iii) of concentrating the digest, and before detecting or titrating step (iv).

11. Method according to claim 2, in which the protein of pathological conformation which is a marker for the UTA is the PrPsc prion protein.

12. Method according to claim 2, in which the step of concentrating before digestion is carried out by centrifugation, preferably by ultracentrifugation.

13. Method according to claim 3, in which the step of concentrating before digestion is carried out by centrifugation, preferably by ultracentrifugation.

14. Method according to claim 11, in which the step of concentrating before digestion is carried out by centrifugation, preferably by ultracentrifugation.

15. Method according to claim 12, in which the concentrating step (i) comprises ultracentrifugation at 140 000 g for 1 hour.

16. Method according to claim 13, in which the concentrating step (i) comprises ultracentrifugation at 140 000 g for 1 hour.

17. Method according to claim 14, in which the concentrating step (i) comprises ultracentrifugation at 140 000 g for 1 hour.

18. Method according to claim 3, in which the concentrating step (iii) is carried out by centrifugation.

19. Method according to claim 4, in which the concentrating step (iii) is carried out by centrifugation.

20. Method according to claim 5, in which the concentrating step (iii) is carried out by centrifugation.
